(19) United States
(12) Patent Application Publication
(10) Pub. No.: US 2026/0363535 A1
(43) Pub. Date: Nov. 19, 2026

(54) DETERMINING A TARGET DISTANCE IN A LASER PROCEDURE

(71) Applicant: GYRUS ACMI, INC. D/B/A OLYMPUS SURGICAL TECHNOLOGIES AMERICA, Westborough, MA (US)

(72) Inventors: Charles A. Baker, Rogers, MN (US); David Bloem, Maple Grove, MN (US); Kester Julian Batchelor, Mound, MN (US); Theodore C. Blus, Arden Hills, MN (US)

(21) Appl. No.: 19/480,803

(22) PCT Filed: May 1, 2024
(86) PCT No.: PCT/US2024/027250

§ 371 Date: Oct. 31, 2025

Related U.S. Application Data
(60) Provisional application No. 63/499,536, filed on May 2, 2023.

(51) Int. Cl. A61B 1/06; A61B 18/00; A61B 18/26

(52) U.S. Cl. CPC A61B 1/0655; A61B 1/0625; A61B 18/26; A61B 2018/00166; A61B 2018/00511; A61B 2018/00642; A61B 2018/00785; A61B 2018/00904; A61B 2018/00982

(57) ABSTRACT

A surgical laser system can include an endoscope. At least one light emitter located on a distal end of the endoscope can direct emitted light toward a target. The target can reflect at least some of the emitted light to form reflected light. At least one camera located on the distal end of the endoscope can capture, with at least some of the reflected light, at least one image of the target illuminated with the emitted light. A controller can determine, from the at least one image, a distance from the distal end of the endoscope to the target. A force sensor can sense when the optical fiber has contacted the target. A controller can cause an actuator to retract the optical fiber proximally by a. specified distance in response to sensing that the optical fiber has contacted the target.

### CROSS-REFERENCE TO RELATED APPLICATION

This application claims the benefit of U.S. Provisional Application No. 63/499,536, filed May 2, 2023, the entirety of which is hereby incorporated by reference.

### FIELD OF THE DISCLOSURE

This document relates generally to endoscopic systems, and more specifically relates to systems and methods for determining and controlling a distance between a tip of a medical device and a target.

### BACKGROUND OF THE DISCLOSURE

An operator, such as a physician, practitioner, or user, can use an endoscope to provide visual access to an internal location of a patient. The operator can insert an endoscope into a patient's body. The endoscope can deliver light to a target being examined, such as a target anatomy or object. The endoscope can collect light that is reflected from the object. The reflected light can carry information about the target being examined.

An endoscope can include a working channel. In some examples, the operator can perform suction through the working channel. In some examples, the operator can pass instruments, such as brushes, biopsy needles or forceps, through the working channel. In some examples, the operator can perform minimally invasive surgery through the working channel, such as to remove unwanted tissue or foreign objects from the body of the patient.

An endoscope can use a laser or plasma system to perform laser therapy, such as ablation, coagulation, vaporization, fragmentation, lithotripsy, and others. In laser therapy, the operator can use the endoscope to deliver surgical laser energy to various target treatment areas, such as soft or hard tissue. In lithotripsy, the operator can use the endoscope to deliver surgical laser energy to break down calculi structures in the patient's kidney, gallbladder, ureter, or other stone-forming regions, or to ablate large calculi into smaller fragments.

### SUMMARY

In an example, a surgical system can include: an endoscope, at least one light emitter disposed at a distal end of the endoscope and configured to illuminate a target site; at least one imaging sensor configured to capture image data associated with the illuminated target site; at least one optical fiber positionable to extend from the distal end of the endoscope and configured to emit fiber light toward the target site; a sensor configured to detect light from the target site; and a controller configured to: determine a distance from a distal end of the at least one optical fiber to the target site based at least in part on the captured image data, determine an optical property associated with the light from the target site; and establish a relationship between the optical property and the determined distance from the distal end of the at least one optical fiber to the target site.

In an example, a method for operating an endoscope, the endoscope including at least one optical fiber positionable to extend from a distal end of the endoscope, can include: illuminating, with at least one light emitter, a target site; capturing, with at least one imaging sensor, image data associated with the illuminated target site; emitting, from the at least one optical fiber, fiber light toward the target site; sensing, with a sensor, light from the target site; determining, with a controller, a distance from a distal end of the at least one optical fiber to the target site based at least in part on the captured image data; determining, with the controller, an optical property associated with the light from the target site; and establishing, with the controller, a relationship between the optical property and the determined distance from the distal end of the at least one optical fiber to the target site.

In an example, a surgical system can include: an endoscope; an optical fiber positionable to extend from a distal end of the endoscope and configured to emit fiber light toward a target; an actuator configured to advance the optical fiber distally and retract the optical fiber proximally with respect to the endoscope; a force sensor configured to sense when the optical fiber has contacted the target; and a controller configured to cause the actuator to retract the optical fiber proximally by a specified distance in response to sensing that the optical fiber has contacted the target.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 shows a side-view schematic drawing of an example of a surgical system.

FIG. 2 shows a perspective view of an example of a configuration at the distal end of the endoscope of the surgical system of FIG. 1.

FIG. 3 shows a perspective view of an example of another configuration at the distal end of the endoscope of the surgical system of FIG. 1, for generating the relationship of FIG. 1.

FIG. 4 shows a perspective view of an example of another configuration at the distal end of the endoscope of the surgical system of FIG. 1, for generating the relationship of FIG. 1.

FIG. 5 shows a side-view schematic drawing of an example of a configuration of the surgical system of FIG. 1, in which the at least one optical fiber can include a single optical fiber.

FIG. 6 shows a side-view schematic drawing of an example of another configuration of the surgical system of FIG. 1, in which the at least one optical fiber can include a first optical fiber and a second optical fiber.

FIG. 7 shows a side-view schematic drawing of an example of a configuration for the fiber sensing and positioning mechanism of the surgical system of FIG. 1.

FIG. 8 shows a flow chart of an example of a method for operating an endoscope.

FIG. 9 shows a flow chart of an example of a method for operating an endoscope.

FIG. 10 shows a flow chart of an example of a method for operating an endoscope.

FIG. 11 shows a schematic diagram of an exemplary computer-based clinical decision support system that is configured to provide a value of distance based on an optical property associated with the light from the target site.

### DETAILED DESCRIPTION

In a laser therapy treatment, a practitioner can position a distal end of an endoscope close to a target, such as a kidney stone. The endoscope can include an optical fiber that can deliver therapeutic laser light to the target, such as via a distal end of the optical fiber. During the treatment, it can be beneficial to dynamically monitor or dynamically control a separation between the distal end of the optical fiber and the target. For example, if the distal end of the optical fiber is positioned too close to the target, it could lead to a condition known as flashing, which can degrade the distal end of the optical fiber. Likewise, if the distal end of the optical fiber is positioned too far from the target, then a significant fraction of the therapeutic laser light can be absorbed before reaching the target, which can decrease an efficiency of the laser therapy treatment or cause the treatment to take longer.

It is found that the amount of reflected light that returns proximally through the optical fiber, or through another optical fiber, optionally with spectral filtering, (referred to as “return light” in the following paragraphs) can vary repeatably as a function of the separation between the distal end of the optical fiber and the target. As a result, a surgical system that includes the endoscope can use the amount of return light to dynamically monitor the separation between the distal end of the optical fiber and the target (referred to as “real-time separation” in the following paragraphs).

Specifically, during a laser therapy treatment, the surgical system can dynamically measure the amount of return light to dynamically determine the real-time separation, and, in response to the real-time separation value, can provide user feedback and/or take an action. For example, the surgical system can provide user feedback representing the real-time separation to the practitioner, such as displaying a numerical value on a display, displaying a graphical representation of the real-time separation on a display, displaying visual indicators that show when the real-time separation is in one of several specified ranges (such as too small, acceptable, too large, and so forth), playing an audio alert, and others. As another example, the surgical system can take an action in response to the real-time separation, such as distally retracting the optical fiber if the real-time separation is too low, automatically positioning the distal end of the optical fiber to have a specified value of real-time separation, or others.

The surgical system can include a calibration technique, which can determine the relationship between the return light and the real-time separation for a particular patient, for a particular instance of insertion of the endoscope. For example, as further described below, the calibration technique can measure values (e.g., intensities or powers) of return light for different values of real-time separation, use the measured values to form a calibration curve, and store the calibration curve. During the laser therapy treatment, the surgical system can measure a value (e.g., intensity or power) of return light, then refer to the calibration curve to determine the real-time separation as a function of the return light value.

The following discussion pertains to performing the calibration technique, which can be performed at the beginning of a laser therapy treatment. After performing the calibration technique, a practitioner can subsequently use the return light to determine the real-time separation during the remainder of the laser therapy treatment.

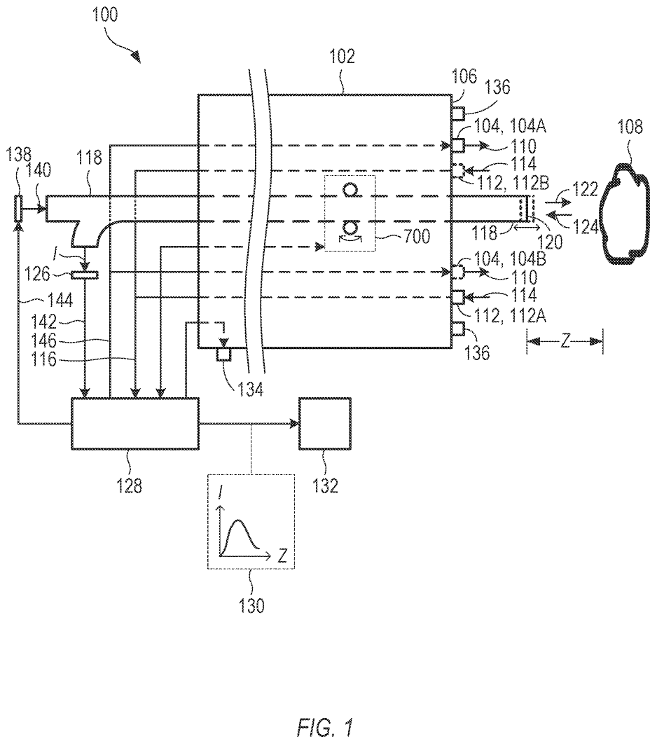

FIG. 1 shows a side-view schematic drawing of an example of a surgical system 100. The configuration of FIG. 1 is but one example of a surgical system; other configurations can also be used.

The surgical system 100 can include an endoscope 102. The endoscope 102 can include an elongated body portion extending between a proximal end and a distal end 106. The precise shape of the elongated body portion can depend on the medical procedure for which the endoscope 102 was originally designed. For simplicity, the elongated body portion is shown as being a cylinder, and having a circular cross-section, taken orthogonal to the direction of elongation. Other suitable shapes can also be used.

The endoscope 102 can include various electrical connections that extend along a length of the endoscope 102, such as to electrically power one or more light sources or light emitters at the distal end 106 of the endoscope 102 and/or to deliver data signals from one or more cameras or imaging sensors at the distal end 106 of the endoscope 102. Although the electrical connections in FIG. 1 are shown as each extending proximally to a respective location at the proximal end of the elongated endoscope 102, in practice, the electrical connections can be grouped together so that the electrical connections can be made with a single connector at or near the proximal end of the endoscope 102. For example, in practice, the electrical connections can extend along a single channel within the endoscope 102 and can fan out as needed at or near the distal end 106 of the endoscope 102.

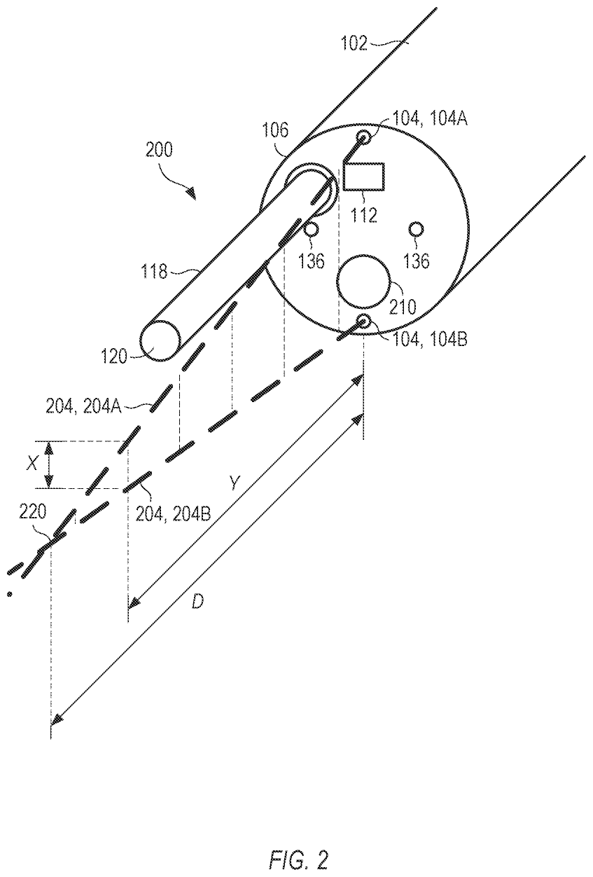

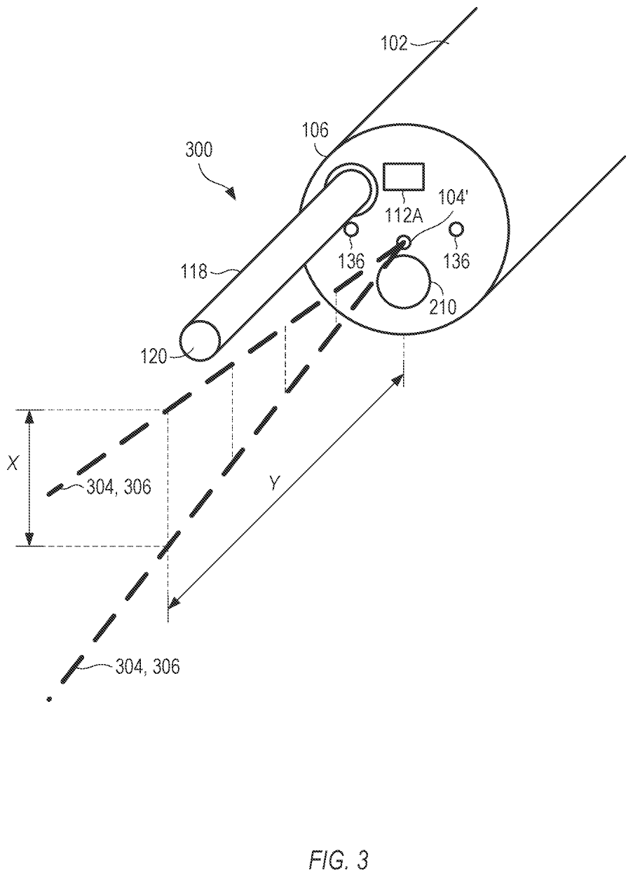

The surgical system 100 can include at least one light emitter 104 disposed at the distal end 106 of the endoscope 102. The at least one light emitter 104 can be configured to illuminate a target site 108. Optionally, the at least one light emitter 104 may direct one or more beams 110 of a specified geometry toward the target site 108. FIGS. 2 and 3 below and the accompanying description pertain to the specified geometry and using the specified geometry to perform a distance measurement.

The surgical system 100 can include at least one imaging sensor 112. The at least one imaging sensor 112 can be configured to capture image data 116 associated with the illuminated target site 108. In some examples, the at least one imaging sensor 112 can capture the image data 116 while the at least one light emitter 104 is illuminating the target site 108 with one or more beams of a specified geometry. In some examples, the at least one imaging sensor 112 can include a camera that is configured to form a video image of the target site 108. In some examples, the at least one imaging sensor 112 can form at least one image from light 114 that is emitted by the at least one light emitter 104 and reflected by the target site 108.

In some examples, the camera used in the calibration technique can be the same camera that is subsequently used during the laser therapy treatment to form a real-time video image of the target site 108, such as to provide a live image of the target site 108 for the practitioner during the treatment procedure. To provide illumination for the real-time video image during the treatment procedure, the surgical system 100 can also include one or more white-light light sources 136, such as one or more white-light light-emitting diodes (LEDs), located at the distal end 106 of the endoscope 102. In some examples, the one or more white-light light sources 136 can be used during the treatment procedure and may not be used during the calibration prior to the treatment procedure.

The surgical system 100 can include at least one optical fiber 118. The at least one optical fiber 118 can be positionable to extend from the distal end 106 of the endoscope 102. The at least one optical fiber 118 can be configured to emit fiber light 122 toward the target site 108. In some examples, the fiber light 122 can be used during the treatment procedure, such as to be directed onto a kidney stone to perform dusting and/or fragmenting of the kidney stone.

The surgical system 100 can include a laser light source 138 configured to generate laser light 140, direct the laser light 140 into a proximal portion of the optical fiber 118, and direct the laser light 140 distally along a length of the optical fiber 118 to emerge from a distal end of the optical fiber 118 to form the fiber light 122. In some examples, the laser light source 138 can include a thulium fiber laser, which can produce light at a wavelength of 1908 nm and/or 1940 nm. In some examples, the laser light source 138 can include a thulium: YAG (yttrium aluminum garnet) laser, which can produce light at a wavelength of 2010 nm. In some examples, the laser light source 138 can include a holmium: YAG laser, which can produce light at a wavelength of 2120 nm. In some examples, the laser light source 138 can include an erbium: YAG laser, which can produce light at a wavelength of 2940 nm. For these (and other) laser light sources, the laser light 140 has a wavelength or wavelengths in a portion of the electromagnetic spectrum at which water (a major component of tissue) has a relatively high absorption. During a procedure, the tissue can absorb the laser light, can heat locally to a relatively high temperature, and can break apart due to local thermal strains within the tissue.

The surgical system 100 can include a sensor 126. The sensor 126 can be configured to detect light 124 from the target site 108. In some examples, the distal end 120 of the at least one optical fiber 118 can emit the fiber light 122 toward the target site 108 and couple at least some of the light 124 reflected from the target site 108 into the at least one optical fiber 118 to form return light. In some examples, the sensor 126 can comprise a spectrometer located separate from the endoscope 102 and configured to measure an optical power/intensity (I) of the return light. In some examples, the sensor 126 can be located at or near a proximal portion of the at least one optical fiber 118. In some examples, the sensor 126 can include a single (e.g., non-pixilated) detector area, such that the sensor 126 is a non-imaging sensor. The sensor 126 can produce a sensor signal 142 in response to the received optical power/intensity (I) of the return light.

The surgical system 100 can include a controller 128. The controller 128 can receive image data 116 from the at least one imaging sensor 112. The controller 128 can receive the sensor signal 142 from the sensor 126. The controller 128 can produce a laser control signal 144 to control the laser light source 138. The controller 128 can produce at least one light emitter control signal 146 to control the at least one light emitter 104 and/or the white-light light sources 136.

The controller 128 can be configured to determine a distance (Z) from a distal end 120 of the at least one optical fiber 118 to the target site 108 based at least in part on the captured image data 116.

The controller 128 can be further configured to determine an optical property associated with the light 124 from the target site 108. In some examples, the optical property can comprise the optical power/intensity (I) of the return light. In some examples, the optical can comprise the optical power/intensity (I) of one or more spectral regions of the return light.

The controller 128 can be further configured to establish a relationship 130 between the optical property (e.g., power or intensity) of the return light and the determined distance (Z) from the distal end 120 of the at least one optical fiber 118 to the target site 108. In some examples, the relationship 130 can include a calibration curve that includes measurements of the optical power/intensity (I) of the light 124 from the target site 108 as a function of distance (Z) from the distal end 120 of the at least one optical fiber 118 to the target site 108, for a plurality of distances (Z) from the distal end 120 of the at least one optical fiber 118 to the target site 108. The relationship 130, such as the calibration curve that relates the distance (Z) to the optical power/intensity (I), can be a final product of the calibration technique described herein. In some examples, the controller 128 can save the relationship 130 to a storage device 132, such as lookup table. When saved, the relationship 130 can subsequently be accessed by the controller 128 or other suitable processor, such as to generate a value of a real-time distance (Z) in response to a measured real-time of optical power/intensity (I).

In some examples, the controller 128 can be further configured to, based at least in part on the relationship 130 such as the calibration curve, dynamically determine a real-time separation between the distal end 120 of the at least one optical fiber 118 and the target site 108 in response to a real-time measured value of the optical power/intensity of the light 124 from the target site 108.

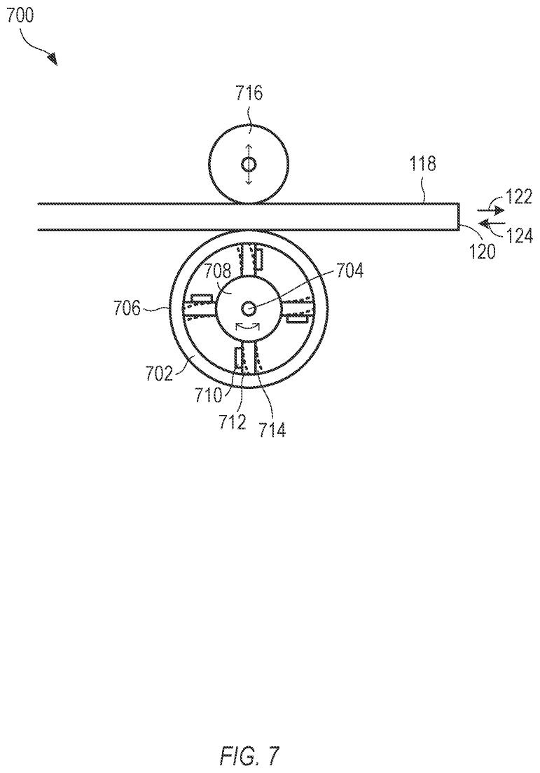

In some examples, the controller 128 can be further configured to suggest movement (e.g., advancing or retraction) of the optical fiber and/or a degree of movement (e.g., 2 mm) for increasing treatment efficiency. For example, the controller 128 can measure an optical power/intensity (I), determine from the measured optical power/intensity (I) where the optical fiber is positioned with respect to the target site 108, and instruct the actuator to move the optical fiber to a location that corresponds to a peak of the calibration curve. As another example, the controller 128 can measure an optical power/intensity (I), determine from the measured optical power/intensity (I) where the optical fiber is positioned with respect to the target site 108, and instruct the actuator to move the optical fiber by a specified amount (e.g., 1 mm) toward the location that corresponds to a peak of the calibration curve. In addition, the controller 128 may be further configured to automatically position the distal end 120 of the at least one optical fiber 118, using the calibration curve, to set the real-time separation between the distal end 120 of the at least one optical fiber 118 and the target site 108 to a specified value. For example, the surgical system 100 can include a fiber sensing and positioning mechanism 700 that can vary a position of the at least one optical fiber 118 in the distal and the proximal directions and can sense when a distal end of the at least one optical fiber 118 has contacted the target site 108. FIG. 7 below provides additional details regarding the fiber sensing and positioning mechanism 700.

In some examples, the controller 128 can be further configured to provide user feedback representing the real-time separation between the distal end of the at least one optical fiber and the target site. For examples, the surgical system 100 can include at least one alarm 134, that can provide an alert to the practitioner in response to the real-time distance (Z) satisfying a specified criterion or specified criteria. For example, when the real-time distance (Z) falls below a specified threshold, meaning that the distal end of the at least one optical fiber 118 can be too close to the target site 108, the at least one alarm 134 can produce an alarm to alert the practitioner. As another example, when the real-time distance (Z) exceeds a specified threshold, meaning that the distal end of the at least one optical fiber 118 can be too far from the target site 108, the at least one alarm 134 can produce an alarm to alert the practitioner. As still another example, when the real-time distance (Z) exits a specified acceptable range of distances, the at least one alarm 134 can produce an alarm to alert the practitioner. In some examples, the at least one alarm 134 can include a speaker that can produce a sound, such as a beep, to alert the practitioner. In some examples, the speaker can produce a first sound, such as low-pitched beep, when the real-time distance (Z) is greater than a greatest value of a specified range of distances, and a second sound, such as high-pitched beep, when the real-time distance (Z) is less than a least value of the specified range of distances. In some examples, the at least one alarm 134 can include one or more lights, such as one or more light-emitting diodes, such as one or more differently colored light-emitting diodes, to provide the alert. Other suitable alarms can also be used.

FIG. 2 shows a perspective view of an example of a configuration 200 at the distal end 106 of the endoscope 102 of the surgical system 100 of FIG. 1, for generating the relationship 130 of FIG. 1. The configuration 200 of FIG. 2 is but one example of a suitable configuration; other suitable configurations can also be used.

In the configuration 200 of FIG. 2, the at least one light emitter 104 can include two light emitters 104A, 104B disposed at the distal end 106 of the endoscope 102. In some examples, each light emitter 104 can emit a respective beam 204, such as a collimated beam. In some examples, the beam 204 can have a cross-sectional intensity profile that has a peak and decreases at increasing distances away from the peak. In other examples, the beam 204 can have a cross-sectional intensity profile that is uniform or approximately uniform over the extent of the beam. Other suitable beam profiles can also be used. In some examples, the light emitters 104A, 104B emit respective beams 204A, 204B having the same wavelength. In other examples, the light emitters 104A, 104B emit respective beams 204A, 204B having different wavelengths. In some examples, the light emitters 104A, 104B emit respective beams 204A, 204B having different spectral profiles. In some examples, using different spectral profiles can allow for more complex multidimensional interference patterns.

In some examples, the two light emitters 104A, 104B can illuminate the target site 108 with respective beams 204A, 204B that are angled with respect to each other. In some examples, the respective beams 204A, 204B can cross at a predetermined distance (D) away from the distal end 106 of the endoscope 102, such as at location 220.

In some examples, the at least one imaging sensor 112 can include a single camera. In some examples, the controller 128 (FIG. 1) can determine the distance (Z) in part by determining, from the captured image data 116, a separation (X) between the beams 204A, 204B.

For example, at least one imaging sensor 112 can capture an image of the target site 108 and direct the image to the controller as the captured image data 116. In one embodiment, when the capture image depicts a single point corresponding to the cross point of the two beams 204A, 204B, it indicates that the image plane of the sensor 112 is at the predetermined distance (D) away from the distal end 106. In various embodiments, the captured image depicts two points corresponding to the two respective beams, the controller 128 may compute the distance between the image plane and the distal end 106 based on geometry. For example, the controller 128 can measure the separation (X) between the beams 204A, 204B in the image. The controller 128 can use the measured separation (X), a specified lateral separation between the light emitters 104A, 104B, and a specified angle between the beams 204A, 204B to determine a distance (Y) between the distal end 106 of the endoscope 102 and the target site 108. The controller can subtract a distance between the distal end 106 of the endoscope 102 and the distal end 120 of the at least one optical fiber 118 from the distance (Y) to determine the distance (Z) between the distal end 120 of the at least one optical fiber 118 and the target site 108. This is but one example of a technique for determining the determining the distance (Z) from the captured image data 116; other techniques can also be used.

In various embodiments, while the at least one imaging sensor 112 captures images of the target site (and, in one embodiment, the controller 128 is configured to compute the distance (Z) between the distal end 120 of the at least one optical fiber 118 and the target site 108), the sensor 126 (e.g., a spectrometer) may simultaneously or substantially simultaneously measure the optical power/intensity (I) of return light from the target site 108. Exemplary approaches for measuring the optical power/intensity (I) of return light from the target site 108 may be found in U.S. Patent Application Publication No. 2021/0038306, the content of which is incorporated herein by reference. The determined distance (Z) and the measured optical power/intensity (I) of return light from the can then be used to generate the calibration curve 130 indicating a relationship between the distance (Z) and the optical power/intensity (I) of the return light.

The endoscope 102 can include a suction channel 210, which can extend longitudinally along the endoscope 102 from a proximal portion of the endoscope to the distal end 106 of the endoscope 102. The suction channel 210 can be connected to a suction-providing device, which can remove debris and portions of the target site 108 through the suction channel 210 during the laser therapy treatment. The optical fiber 118 may be inserted into the suction channel 210 for delivering laser light to the target site 108 for treatment. Alternatively, the optical fiber 118 may be inserted in the endoscope 102 via a second channel different from the suction channel 210.

FIG. 3 shows a perspective view of an example of another configuration 300 at the distal end 106 of the endoscope 102 of the surgical system 100 of FIG. 1, for generating the relationship 130 of FIG. 1. The configuration 300 of FIG. 3 is but one example of a suitable configuration; other suitable configurations can also be used.

In the configuration 300 of FIG. 3, the at least one light emitter 104 can include a single light emitter 104′ disposed at the distal end 106 of the endoscope 102. In some examples, the single light emitter 104′ can illuminate the target site 108 with a beam 304 that diverges away from the single light emitter 104A with a controlled, specified divergence angle (using, for example, an optically designed component).

The specified divergence angle can be formed between opposing edges 306 of the diverging beam 304. In FIG. 3, the edges 306 of the diverging beam 304 are represented as dashed lines. As the diverging beam 304 propagates away from the light emitter 104′ (in a longitudinal direction), a lateral distance (e.g., orthogonal to the longitudinal direction) between the edges 306 increases. In some examples, the edges 306 can be “soft” edges to the diverging beam 304, such as a full-width-at-half-maximum or other suitable fraction of a peak intensity. In other examples, the edges 306 can be “hard” edges, such as limited by a physical aperture. For example, the edge of the diverging beam 304 may form a circle or any shape on the image captured by the at least one imaging sensor 112. Other suitable beam profiles can also be used.

In some examples, the controller 128 can be configured to determine the distance (Z) in part by determining, from the captured image data 116, a cross-sectional size of the beam 304. For example, the camera 112A can capture an image of the target site 108 and direct the image to the controller as the captured image data 116. The controller 128 can measure the separation (X) between the edges 306 of the diverging beam 304 in the image (e.g., the diameter of the circle on the captured image). The controller 128 can use the measured separation (X) and a specified divergence angle of the beam 304 to determine a distance (Y) between the distal end 106 of the endoscope 102 and the target site 108. The controller can subtract a distance between the distal end 106 of the endoscope 102 and the distal end 120 of the at least one optical fiber 118 from the distance (Y) to determine the distance (Z) between the distal end 120 of the at least one optical fiber 118 and the target site 108. This is but one example of a technique for determining the determining the distance (Z) from the captured image data 116; other techniques can also be used. For example, any optical components that can be used to generate any shapes of the divergent beam on the captured images may be implemented for measuring the distance (Z) using the approach described above.

Again, while the camera 112A captures images of the target site (and, in one embodiment, the controller is configured to compute the distance (Z) between the distal end 120 of the at least one optical fiber 118 and the target site 108), the sensor 126 (e.g., a spectrometer) may simultaneously or substantially simultaneously measure the optical power/intensity (I) of return light from the target site 108. Based on the determined distance (Z) and the measured optical power/intensity (I) of return light from the target site 108, the calibration curve 130 indicating a relationship between the distance (Z) and the optical power/intensity (I) of the return light can be generated.

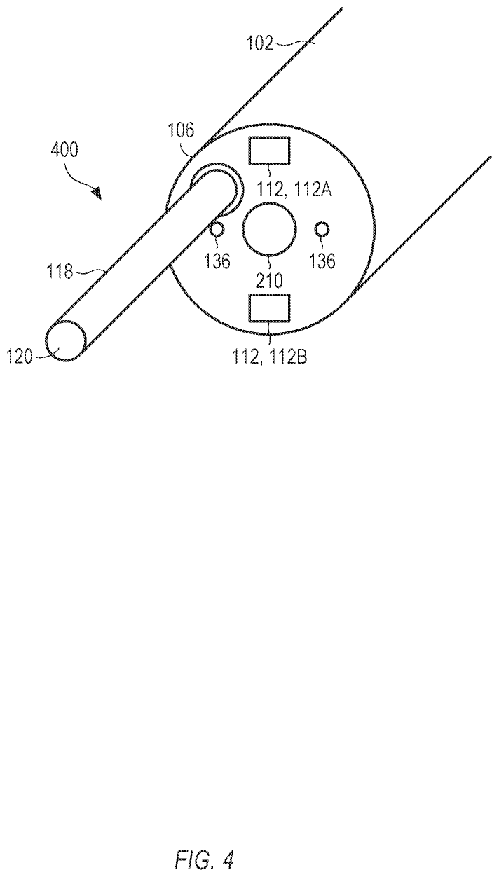

FIG. 4 shows a perspective view of an example of another configuration 400 at the distal end 106 of the endoscope 102 of the surgical system 100 of FIG. 1, for generating the relationship 130 of FIG. 1. The configuration 400 of FIG. 4 is but one example of a suitable configuration; other suitable configurations can also be used.

In some examples, the at least one imaging sensor 112 can include a first camera 112A and a second camera 112B that are disposed at the distal end 106 of the endoscope 102. In some examples, using two cameras can produce a three-dimensional image. In some examples, using a quantity N cameras can produce a quantity N+1 dimensional image. In some examples, the first camera 112A and the second camera 112B are separated in a lateral direction.

In some examples, the captured image data 116 can include a first image captured by the first camera 112A and a second image captured by the second camera 112B. In some examples, the first and second images are captured simultaneously, or nearly simultaneously, such that the tissue or material at and around the target site 108 does not move significantly between the times at which the first and second images are captured. The one or more white-light light sources 136 can be activated to produce white light illumination to illuminate the target site 108 while the first and second images are captured.

In some examples, the controller 128 can determine the distance via triangulation from the first image and the second image. For example, the lateral separation between an element in the first image and the corresponding element in the second image can be proportional to a longitudinal distance between the element and the first and second cameras 112A, 112B. This is but one example of a technique for determining the determining the distance (Z) from the captured image data 116; other techniques can also be used.

Similar to another approaches described in FIGS. 2 and 3, while the first and second cameras 112A, 112B capture images (and, in one embodiment, the controller is configured to compute the distance (Z) between the distal end 120 of the at least one optical fiber 118 and the target site 108), the sensor 126 (e.g., a spectrometer) may simultaneously or substantially simultaneously measure the optical power/intensity (I) of return light from the target site 108. Based on the determined distance (Z) and the measured optical power/intensity (I) of return light from the target site 108, the calibration curve 130 indicating a relationship between the distance (Z) and the optical power/intensity (I) of the return light can be generated.

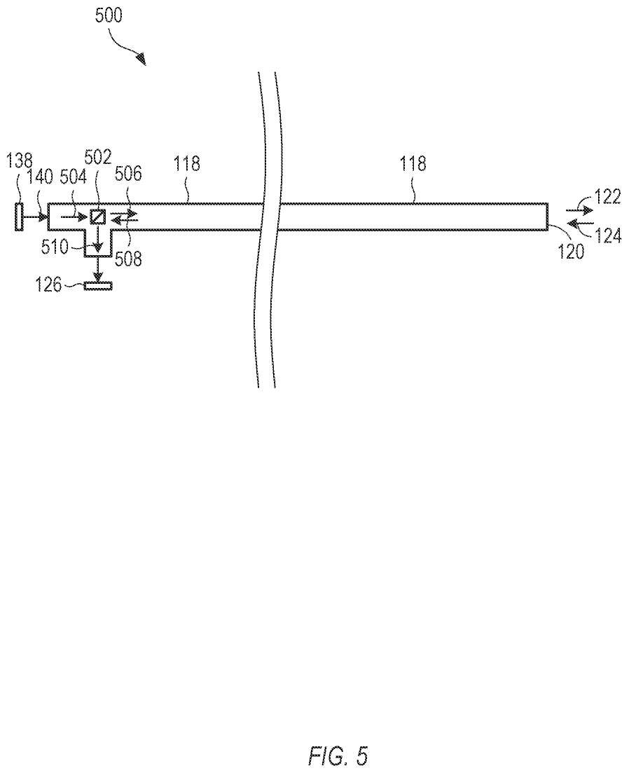

FIG. 5 shows a side-view schematic drawing of an example of a configuration 500 of the surgical system 100 of FIG. 1, in which the at least one optical fiber 118 can include a single optical fiber. The configuration of FIG. 5 is but one example of a surgical system; other configurations can also be used.

In the single-fiber configuration, the laser light source 138 can generate laser light 140, direct the laser light 140 into a proximal portion of the optical fiber 118, and direct the laser light 140 distally along a length of the optical fiber 118 to emerge from the distal end 120 of the optical fiber 118 to form the fiber light 122. The fiber light 122 can reflect from the target site 108 to form a portion of the light 124.

Additionally or alternatively, light illuminated from the one or more white-light LEDs to the target site can be reflected from the target site to form at least a portion of the light 124. At least some of the light 124 can couple into the distal end 120 of the optical fiber 118 and can propagate proximally along the optical fiber 118 as return light 508.

In some examples, the surgical system 100 can further include a beamsplitter 502 configured to separate an optical path of the fiber light 122 from an optical path of the return light 508. For example, the laser light 140 can enter the optical fiber 118 as incident light 504. At least some of the incident light 504 can pass through the beamsplitter 502 to form distally propagating light 506. The distally propagating light 506 can exit the distal end 120 of the optical fiber 118 to form the fiber light 122. At least some of the return light 508 can reflect from the beamsplitter 502 to form outward propagating light 510. At least some of the outward propagating light 510 can strike the sensor 126. In some examples, the location of the laser light source 138 and the sensor 128 can be swapped. In some examples, the beamsplitter 502 can include an at least partially reflective element that is angled with respect to the optical paths that traverse the beamsplitter 502. In some examples, the beamsplitter 502 can be implemented as a fiber splitter.

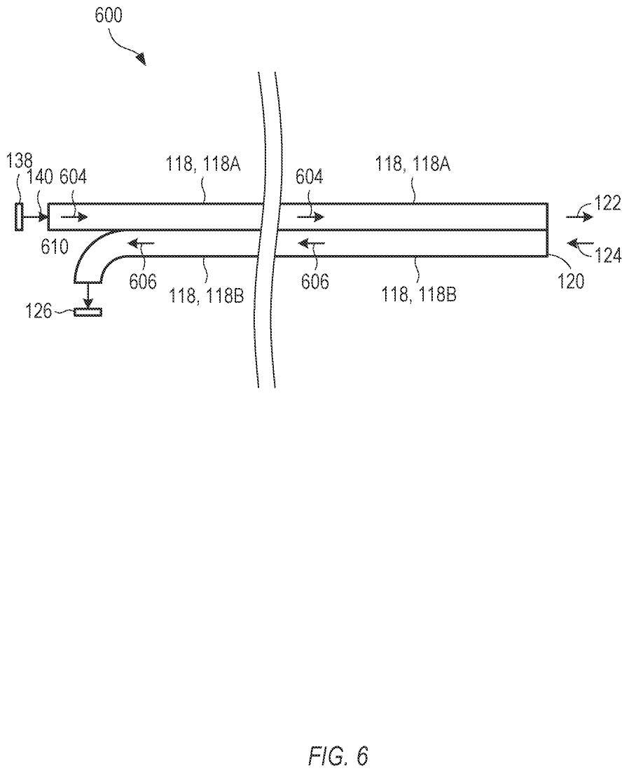

FIG. 6 shows a side-view schematic drawing of an example of another configuration 600 of the surgical system 100 of FIG. 1, in which the at least one optical fiber 118 can include a first optical fiber 118A and a second optical fiber 118B. In some examples, the first optical fiber 118A and the second optical fiber 118B extend generally parallel to each other along a length of the endoscope 102. In some examples, the first optical fiber 118A and the second optical fiber 118B have respective distal ends that are co-planar. In some examples, the first optical fiber 118A and the second optical fiber 118B have respective distal ends that are co-planar and generally orthogonal to a longitudinal axis of the at least one optical fiber 118. The configuration of FIG. 6 is but one example of a surgical system; other configurations can also be used.

In some examples, the first optical fiber 118A can direct the fiber light 122 distally along the first optical fiber 118A toward the target site 108. In some examples, the second optical fiber 118B can direct the return light proximally along the second optical fiber to the sensor 126, such as the spectrometer.

For example, the laser light 140 can enter the first optical fiber 118A as incident light 604. The incident light 604 can exit the distal end 120 of the first optical fiber 118A to form the fiber light 122. At least some of the fiber light 122 can reflect from the target site 108 to form the light 124. Additionally or alternatively, light illuminated from the one or more white-light LEDs to the target site can be reflected from the target site to form at least a portion of the light 124. At least some of the light 124 can enter a distal end of the second optical fiber 118B and propagate proximally along the second optical fiber 118B as proximally propagating light 606. At least some of the proximally propagating light 606 can strike the sensor 126.

In some examples, the location of the laser light source 138 and the sensor 128 can be swapped. In some examples, the beamsplitter 502 can include an at least partially reflective element that is angled with respect to the optical paths that traverse the beamsplitter 502. In some examples, the beamsplitter 502 can be implemented in free space, such that the light incident on and reflected from the beamsplitter propagates in free space. In other examples, the beamsplitter 502 can be implemented as a fiber splitter, such that the light incident on and reflected from the beamsplitter propagates in an optical fiber.

FIG. 7 shows a side-view schematic drawing of an example of a configuration for the fiber sensing and positioning mechanism 700 of the surgical system 100 of FIG. 1. The configuration of FIG. 7 is but one example of a suitable fiber sensing and positioning mechanism; other suitable configurations can also be used.

For example, the surgical system 100 can include an endoscope 102 (FIG. 1). The surgical system 100 can further include an optical fiber 118 positionable to extend from a distal end 106 of the endoscope 102 (FIG. 1) and configured to emit fiber light 122 toward a target, such as target site 108. The surgical system 100 can further include an actuator configured to advance the optical fiber 118 distally and retract the optical fiber 118 proximally with respect to the endoscope 102. The surgical system 100 can further include a force sensor configured to sense when the optical fiber 118 has contacted the target. The surgical system 100 can further include a controller 128 configured to cause the actuator to retract the optical fiber 118 proximally by a specified distance in response to sensing that the optical fiber 118 has contacted the target.

In various embodiments, when the force sensor detects that the tip of the optical fiber 118 has contacted the target, the controller sets the position of the fiber tip as position “0,” namely, the distance between the fiber tip and the target is zero. From that point, the optical fiber 118 can be retracted proximally in a controlled manner using the actuator. For example, the actuator may be configured to precisely control movement of the optical fiber 118; as a result, the distance (Z) between the tip of the optical fiber 118 during retraction of the optical fiber 118 can be determined. In addition, while the actuator is configured to advance or retract the optical fiber 118 (and, in one embodiment, the controller is configured to compute the distance (Z) between the distal end 120 of the at least one optical fiber 118 and the target site 108 based on the recorded position “0” and the movement of the optical fiber 118), the sensor 126 (e.g., a spectrometer) may simultaneously or substantially simultaneously measure the optical power/intensity (I) of return light from the target. Based on the determined distance (Z) and the measured optical power/intensity (I) of return light from the target, the calibration curve 130 indicating a relationship between the distance (Z) and the optical power/intensity (I) of the return light can be generated.

In some examples, the actuator can include a wheel 702. The wheel 702 can have a center 704 that is fixed in position with respect to the endoscope 102. The wheel 702 can have a circumferential surface 706 that contacts the optical fiber 118. The wheel 702 can be rotatable from a rotary actuator 708 located proximate a center of the wheel 702.

In some examples, the force sensor can include a torque sensor disposed between the rotary actuator and the circumferential surface 706. In some examples, the torque sensor can be implemented as a strain sensor 710 disposed along a generally radial flexure 712 that extends between the rotary actuator 708 and the circumferential surface 706. During typical use, the generally radial flexure 712 can be straight or nearly straight, optionally along a radial direction. When the distal end 120 of the optical fiber 118 has contacted a target, such as the target site 108, the generally radial flexure 712 can deform in the tangential direction (such as to form deformed flexure 714), and the strain sensor 710 can sense a non-zero value of strain. The controller 128 can optionally convert the strain value to a torque value by multiplying by a radius of the circumferential surface 706. The fiber sensing and positioning mechanism 700 can also include a guide wheel 716 positioned such that the optical fiber 118 extends between the guide wheel 716 and the circumferential surface 706. During use, the guide wheel 716 can apply a resisting force, through the optical fiber 118, to the wheel 702, to ensure that the wheel 702 remains in contact with the optical fiber 118 without slipping. The guide wheel 716 can optionally have an adjustable position to account for different diameters of the optical fiber 118 or plurality of optical fibers 118.

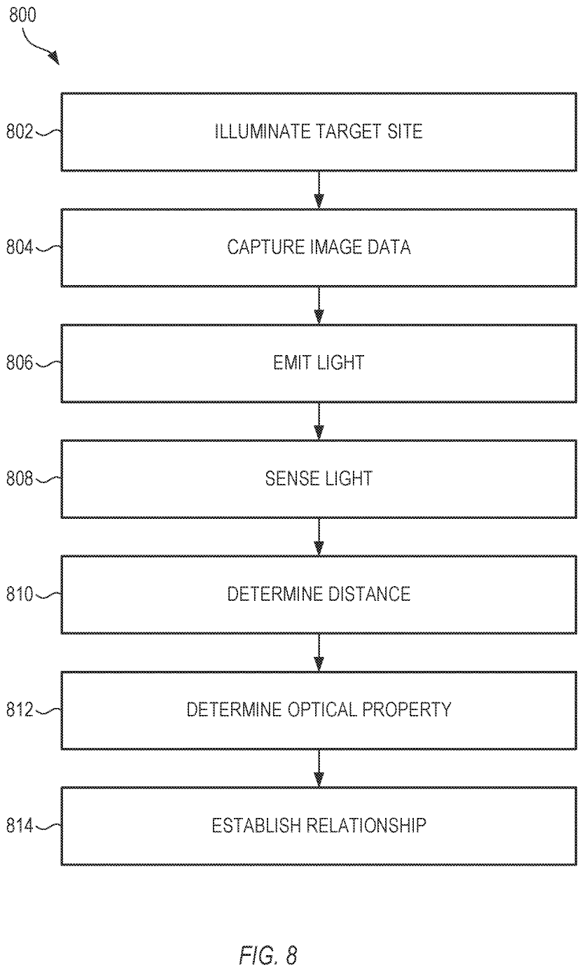

FIG. 8 shows a flow chart of an example of a method 800 for operating an endoscope, such as endoscope 102 (FIG. 1). The endoscope can include at least one optical fiber, such as at least one optical fiber 118, positionable to extend from a distal end of the endoscope.

At operation 802, the endoscope can illuminate, with at least one light emitter, such as at least one light emitter 104, a target site, such as target site 108.

At operation 804, the endoscope can capture, with at least one imaging sensor, such as at least one imaging sensor 112, image data associated with the illuminated target site.

At operation 806, the endoscope can emit, from the at least one optical fiber, fiber light toward the target site. Additionally or alternatively, the endoscope can emit light from the LEDs toward the target site.

At operation 808, the endoscope can sense, with a sensor, such as sensor 126, return light from the target site.

At operation 810, the endoscope can determine, with a controller, such as controller 128, a distance from a distal end of the at least one optical fiber to the target site based at least in part on the captured image data.

At operation 812, the endoscope can determine, with the controller, an optical property (e.g., intensity or power) associated with the light from the target site; and

At operation 814, the endoscope can establish, with the controller, a relationship, such as relationship 130, between the optical property and the determined distance from the distal end of the at least one optical fiber to the target site.

In some examples, the at least one light emitter can include two light emitters disposed on the distal end of the endoscope and configured to emit respective beams that are angled with respect to each other. In some examples, determining the distance can include determining, from the captured image data, a separation between the beams.

In some examples, the at least one light emitter can include a single light emitter disposed on the distal end of the endoscope and configured to emit a beam that diverges away from the single light emitter with a specified divergence angle. In some examples, determining the distance can include determining, from the captured image data, a cross-sectional size of the beam.

In some examples, the at least one imaging sensor can include a first camera and a second camera that are disposed at the distal end of the endoscope. In some examples, the captured image data can include a first image captured by the first camera and a second image captured by the second camera. In some examples, determining the distance can include using triangulation from the first image and the second image.

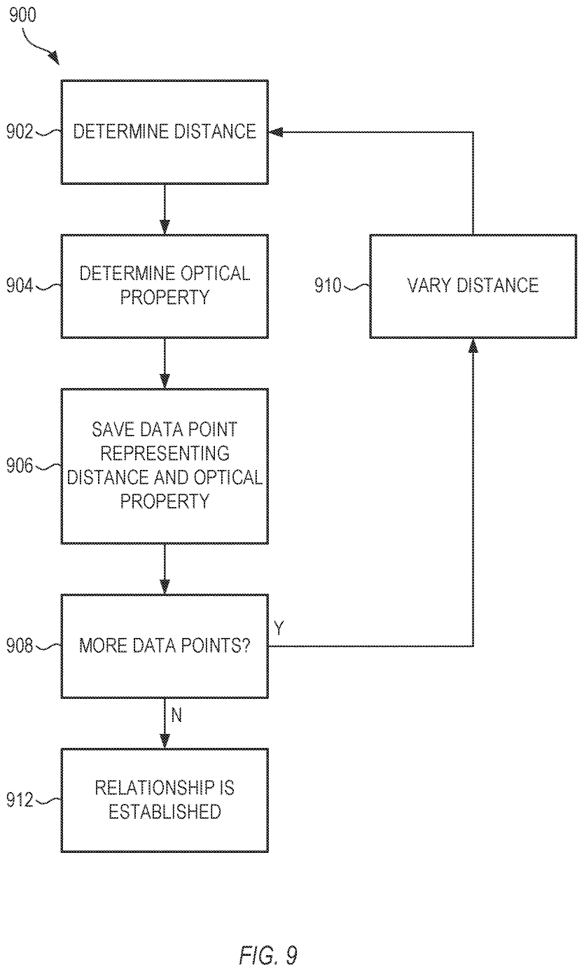

FIG. 9 shows a flow chart of an example of a method 900 for operating an endoscope, such as endoscope 102 (FIG. 1). The endoscope can include at least one optical fiber, such as at least one optical fiber 118, positionable to extend from a distal end of the endoscope. In particular, the method 900 may be suitable for performing a calibration of the endoscope, such as at the beginning of a medical procedure. For example, the method 900 may be performed once to establish the relationship, such as the relationship 130 (FIG. 1). The relationship can include the calibration curve. After the method 900 has been performed, the practitioner can proceed with the medical procedure. During the medical procedure, the calibration curve can function as a lookup table, and can provide values of distance (Z) as needed.

At operation 902, the distance (Z) can be determined, such as by executing the method 800.

At operation 904, the optical property can be determined, such as by executing the method 800.

At operation 906, the controller, such as controller 128, can save a data point representing the determined distance and the optical property. The data point can correspond to one data point on the calibration curve.

At operation 908, the controller can determine whether additional data points can be used to form the full calibration curve. For example, the controller can use a plurality of specified distances, such as equally spaced distances over a specified range of distances, to form the calibration curve. As another example, the controller can use the optical property values from operation 904 to determine whether the calibration curve has peaked, and can take additional data as needed to ensure that the peak value has been included in the calibration curve. If additional data points are needed, the controller can instruct the actuator to vary the distance in operation 910. If no additional data points are needed, the controller can proceed to operation 912, at which the relationship has been established. At operation 912, based on the determined distance (Z) and the measured optical power/intensity (I) of return light from the target, the calibration curve indicating a relationship between the distance (Z) and the optical power/intensity (I) of the return light has been generated.

In some examples, the method 900 can optionally include interpolating between the measured data points, fitting a curve to the measured data points, and/or smoothing the measured data points.

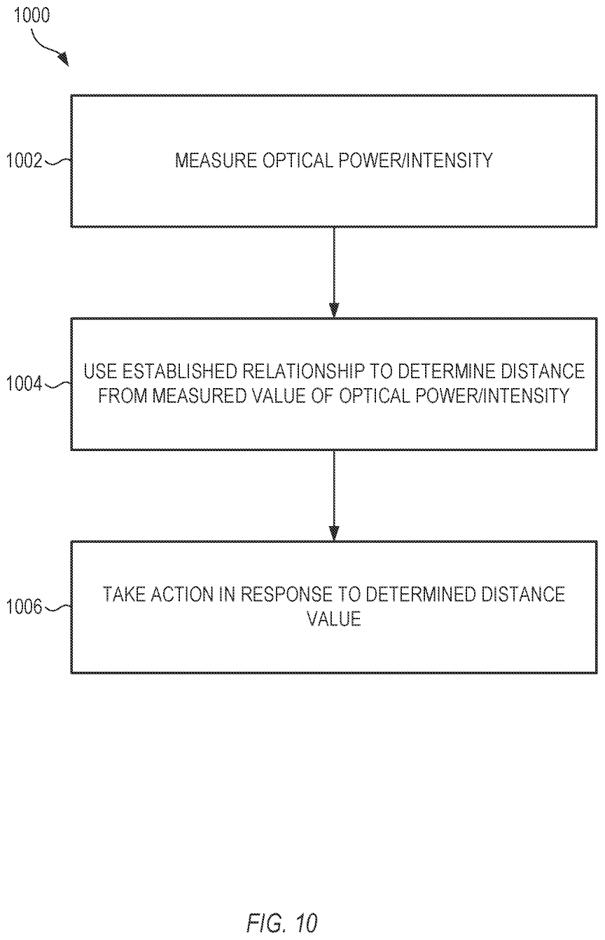

FIG. 10 shows a flow chart of an example of a method 1000 for operating an endoscope, such as endoscope 102 (FIG. 1). The endoscope can include at least one optical fiber, such as at least one optical fiber 118, positionable to extend from a distal end of the endoscope. In particular, the method 1000 may be suitable for execution during the medical procedure, after the endoscope has been calibrated, such as by execution of the method 900.

At operation 1002, a real-time value of optical power/intensity (I) can be measured, such as by the sensor 126 (FIG. 1).

At operation 1004, the established relationship, such as the calibration curve, can be used to determine a real-time value of distance (Z) from the measured real-time value of optical power/intensity (I).

At operation 1006, an action can be taken in response to the determined real-time value of distance (Z). For example, the action can include automatically positioning the distal end of the at least one optical fiber, using the calibration curve, to set the real-time separation between the distal end of the at least one optical fiber and the target site to a specified value. As another example, the action can include providing user feedback representing the real-time separation between the distal end of the at least one optical fiber and the target site. Other suitable actions can also be used.

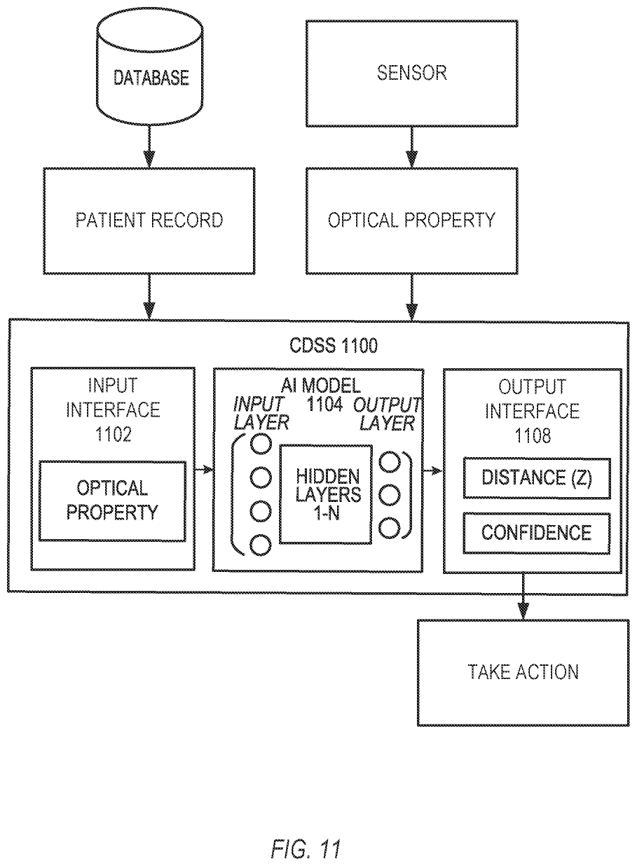

FIG. 11 shows a schematic diagram of an exemplary computer-based clinical decision support system (CDSS) 1100 that is configured to provide a value of distance (Z) based on an optical property associated with the light from the target site.

For example, the optical property can include the optical power/intensity (I) of the return light. In various embodiments, the CDSS 1100 includes an input interface 1102 through which the optical property which is specific to a patient is provided as input features to an artificial intelligence (AI) model 1104, a processor such as controller 128 which performs an inference operation in which the optical property is applied to the AI model to generate the value of distance (Z), and a user interface (UI) through which value of distance (Z) is communicated to a user, e.g., a clinician.

In some embodiments, the input interface 1102 may be a direct data link between the CDSS 1100 and one or more medical devices, such as surgical system 100 or endoscope 102, which generate at least some of the input features. For example, the input interface 1102 may transmit the optical property directly to the CDSS during a therapeutic and/or diagnostic medical procedure. Additionally, or alternatively, the input interface 1102 may be a classical user interface that facilitates interaction between a user and the CDSS 1100. For example, the input interface 1102 may facilitate a user interface through which the user may manually enter the optical property. Additionally, or alternatively, the input interface 1102 may provide the CDSS 1100 with access to an electronic patient record from which one or more input features may be extracted. In any of these cases, the input interface 1102 is configured to collect the optical property in association with a specific patient on or before a time at which the CDSS 1100 is used to assess the medical condition addressed by the surgical system 100 or endoscope 102, such as a kidney stone.

Based on one or more of the above input features, the processor such as controller 128 performs an inference operation using the AI model to generate the value of distance (Z). For example, input interface 1102 may deliver the optical property into an input layer of the AI model which propagates this input feature through the AI model to an output layer. The AI model can provide a computer system the ability to perform tasks, without explicitly being programmed, by making inferences based on patterns found in the analysis of data. AI model explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model may be trained continuously or periodically prior to performance of the inference operation by the processor such as controller 128. Then, during the inference operation, the patient specific input features provided to the AI model may be propagated from an input layer, through one or more hidden layers, and ultimately to an output layer that corresponds to the value of distance (Z).

In some examples, the AI model can include a database, which can include data corresponding to a patient. The database can provide a patient record to the CDSS 1100. IN some examples, the AI model can receive an optical property from a sensor, such as sensor 126 or at least one imaging sensor 112.

During and/or subsequent to the inference operation, the value of distance (Z) may be communicated to the user via the user interface (UI) and/or automatically cause an actuator or an alarm connected to the processor to perform a desired action. For example, the processor can cause the actuator to move the optical fiber with respect to the endoscope. Alternatively, the processor can cause the alarm to alert the practitioner.

In some examples, the CDSS 1100 can optionally be used to determine the action taken in response to a value of distance (Z) and/or a value of the optical property such as optical power/intensity (I).

In the foregoing detailed description, the method and apparatus of the present disclosure have been described with reference to specific embodiments thereof. It will, however, be evident that various modifications and changes may be made thereto without departing from the broader spirit and scope of the present disclosure. The present specification and figures are accordingly to be regarded as illustrative rather than restrictive.

To further illustrate the device and related method disclosed herein, a non-limiting list of examples is provided below. Each of the following non limiting examples can stand on its own or can be combined in any permutation or combination with any one or more of the other examples.

In Example 1, a surgical system can comprise: an endoscope; at least one light emitter disposed at a distal end of the endoscope and configured to illuminate a target site; at least one imaging sensor configured to capture image data associated with the illuminated target site; at least one optical fiber positionable to extend from the distal end of the endoscope and configured to emit fiber light toward the target site; a sensor configured to detect light from the target site; and a controller configured to: determine a distance from a distal end of the at least one optical fiber to the target site based at least in part on the captured image data, determine an optical property associated with the light from the target site; and establish a relationship between the optical property and the determined distance from the distal end of the at least one optical fiber to the target site.

In Example 2, the surgical system of Example 1 can optionally be configured such that: the at least one light emitter comprises two light emitters disposed at the distal end of the endoscope; the two light emitters are configured to illuminate the target site with respective beams that are angled with respect to each other; and the controller is configured to determine the distance in part by determining, from the captured image data, a separation between the beams.

In Example 3, the surgical system of any one of Examples 1-2 can optionally be configured such that the respective beams are configured to cross at a predetermined distance away from the distal end of the endoscope.

In Example 4, the surgical system of any one of Examples 1-3 can optionally be configured such that the at least one imaging sensor comprises a single camera.

In Example 5, the surgical system of any one of Examples 1-4 can optionally be configured such that: the at least one light emitter comprises a single light emitter disposed at the distal end of the endoscope; the single light emitter is configured to illuminate the target site with a beam that diverges away from the single light emitter with a specified divergence angle; and the controller is configured to determine the distance in part by determining, from the captured image data, a cross-sectional size of the beam.

In Example 6, the surgical system of any one of Examples 1-5 can optionally be configured such that the at least one imaging sensor comprises a single camera.

In Example 7, the surgical system of any one of Examples 1-6 can optionally be configured such that: the at least one imaging sensor comprises a first camera and a second camera that are disposed at the distal end of the endoscope; the captured image data comprises a first image captured by the first camera and a second image captured by the second camera, and the controller is configured to determine the distance via triangulation from the first image and the second image.

In Example 8, the surgical system of any one of Examples 1-7 can optionally be configured such that: the distal end of the at least one optical fiber is configured to emit the fiber light toward the target site and couple at least some of the light from the target site into the at least one optical fiber to form return light; the optical property comprises an optical power or intensity of the return light; and the sensor comprises a spectrometer located separate from the endoscope and configured to measure the optical power or intensity of the return light.

In Example 9, the surgical system of any one of Examples 1-8 can optionally be configured such that: the at least one optical fiber comprises a single optical fiber, and the surgical system further comprises a beamsplitter configured to separate an optical path of the fiber light from an optical path of the return light.

In Example 10, the surgical system of any one of Examples 1-9 can optionally be configured such that: the at least one optical fiber comprises a first optical fiber and a second optical fiber; the first optical fiber is configured to direct the fiber light distally along the first optical fiber toward the target site; and the second optical fiber is configured to direct the return light proximally along the second optical fiber to the spectrometer.

In Example 11, the surgical system of any one of Examples 1-10 can optionally be configured such that the relationship comprises a calibration curve that includes measurements of the optical power or intensity of the light from the target site as a function of distance from the distal end of the at least one optical fiber to the target site, for a plurality of distances from the distal end of the at least one optical fiber to the target site.

In Example 12, the surgical system of any one of Examples 1-11 can optionally be configured such that the controller is further configured to, based at least in part on the calibration curve, dynamically determine a real-time separation between the distal end of the at least one optical fiber and the target site in response to a real-time measured value of the optical power or intensity of the light from the target site.

In Example 13, the surgical system of any one of Examples 1-12 can optionally be configured such that the controller is further configured to automatically position the distal end of the at least one optical fiber, using the calibration curve, to set the real-time separation between the distal end of the at least one optical fiber and the target site to a specified value.

In Example 14, the surgical system of any one of Examples 1-13 can optionally be configured such that the controller is further configured to provide user feedback representing the real-time separation between the distal end of the at least one optical fiber and the target site.

In Example 15, a method for operating an endoscope, the endoscope including at least one optical fiber positionable to extend from a distal end of the endoscope, the method can comprise: illuminating, with at least one light emitter, a target site; capturing, with at least one imaging sensor, image data associated with the illuminated target site; emitting, from the at least one optical fiber, fiber light toward the target site; sensing, with a sensor, light from the target site; determining, with a controller, a distance from a distal end of the at least one optical fiber to the target site based at least in part on the captured image data; determining, with the controller, an optical property associated with the light from the target site; and establishing, with the controller, a relationship between the optical property and the determined distance from the distal end of the at least one optical fiber to the target site.

In Example 16, the method of Example 15 can optionally be configured such that: the at least one light emitter comprises two light emitters disposed on the distal end of the endoscope and configured to emit respective beams that are angled with respect to each other; and determining the distance comprises determining, from the captured image data, a separation between the beams.

In Example 17, the method of any one of Examples 15-16 can optionally be configured such that: the at least one light emitter comprises a single light emitter disposed on the distal end of the endoscope and configured to emit a beam that diverges away from the single light emitter with a specified divergence angle; and determining the distance comprises determining, from the captured image data, a cross-sectional size of the beam.

In Example 18, the method of any one of Examples 15-17 can optionally be configured such that: the at least one imaging sensor comprises a first camera and a second camera that are disposed at the distal end of the endoscope; the captured image data comprises a first image captured by the first camera and a second image captured by the second camera; and determining the distance comprises using triangulation from the first image and the second image.

In Example 19, a surgical system can comprise: an endoscope; an optical fiber positionable to extend from a distal end of the endoscope and configured to emit fiber light toward a target; an actuator configured to advance the optical fiber distally and retract the optical fiber proximally with respect to the endoscope; a force sensor configured to sense when the optical fiber has contacted the target; and a controller configured to cause the actuator to retract the optical fiber proximally by a specified distance in response to sensing that the optical fiber has contacted the target.

In Example 20, the surgical system of Example 19 can optionally be configured such that: the actuator comprises a wheel, the wheel having a center that is fixed in position with respect to the endoscope, the wheel having a circumferential surface that contacts the optical fiber, the wheel being rotatable from a rotary actuator located proximate the center of the wheel; and the force sensor comprises a torque sensor disposed between the rotary actuator and the circumferential surface.

1.-20. (canceled)

21. A surgical system, comprising:
an endoscope;
two light emitters disposed at a distal end of the endoscope and configured to illuminate a target site with respective beams that are angled with respect to each other;
at least one imaging sensor configured to capture image data associated with the illuminated target site;
at least one optical fiber positionable to extend from the distal end of the endoscope and configured to emit fiber light toward the target site;
a sensor configured to detect light from the target site; and
a controller configured to:
determine a distance from a distal end of the at least one optical fiber to the target site by determining, from the captured image data, a separation between the beams;
determine an optical property associated with the light from the target site; and
establish a relationship between the optical property and the determined distance from the distal end of the at least one optical fiber to the target site.

22. The surgical system of claim 21, wherein the respective beams are configured to cross at a predetermined distance away from the distal end of the endoscope.

23. The surgical system of claim 21, wherein the at least one imaging sensor comprises a single camera.

24. The surgical system of claim 21, wherein:
the distal end of the at least one optical fiber is configured to emit the fiber light toward the target site and couple at least some of the light from the target site into the at least one optical fiber to form return light;
the optical property comprises an optical power or intensity of the return light; and
the sensor comprises a spectrometer located separate from the endoscope and configured to measure the optical power or intensity of the return light.

25. The surgical system of claim 24, wherein:
the at least one optical fiber comprises a single optical fiber; and
the surgical system further comprises a beamsplitter configured to separate an optical path of the fiber light from an optical path of the return light.

26. The surgical system of claim 24, wherein:
the at least one optical fiber comprises a first optical fiber and a second optical fiber;
the first optical fiber is configured to direct the fiber light distally along the first optical fiber toward the target site; and
the second optical fiber is configured to direct the return light proximally along the second optical fiber to the spectrometer.

27. The surgical system of claim 24, wherein the relationship comprises a calibration curve that includes measurements of the optical power or intensity of the light from the target site as a function of distance from the distal end of the at least one optical fiber to the target site, for a plurality of distances from the distal end of the at least one optical fiber to the target site.

28. The surgical system of claim 27, wherein the controller is further configured to, based at least in part on the calibration curve, dynamically determine a real-time separation between the distal end of the at least one optical fiber and the target site in response to a real-time measured value of the optical power or intensity of the light from the target site.

29. The surgical system of claim 28, wherein the controller is further configured to automatically position the distal end of the at least one optical fiber, using the calibration curve, to set the real-time separation between the distal end of the at least one optical fiber and the target site to a specified value.

30. The surgical system of claim 28, wherein the controller is further configured to provide user feedback representing the real-time separation between the distal end of the at least one optical fiber and the target site.

31. A method for operating an endoscope, the endoscope including at least one optical fiber positionable to extend from a distal end of the endoscope, the method comprising:
illuminating, with two light emitters disposed on the distal end of the endoscope, a target site with respective beams that are angled with respect to each other;
capturing, with at least one imaging sensor, image data associated with the illuminated target site;
emitting, from the at least one optical fiber, fiber light toward the target site;
sensing, with a sensor, light from the target site;
determining, with a controller, a distance from a distal end of the at least one optical fiber to the target site by determining, from the captured image data, a separation between the beams;
determining, with the controller, an optical property associated with the light from the target site; and
establishing, with the controller, a relationship between the optical property and the determined distance from the distal end of the at least one optical fiber to the target site.

32. The method of claim 31, wherein the respective beams cross at a predetermined distance away from the distal end of the endoscope.

33. The method of claim 31, wherein the at least one imaging sensor comprises a single camera.

34. The method of claim 31, wherein:
the distal end of the at least one optical fiber emits the fiber light toward the target site and couples at least some of the light from the target site into the at least one optical fiber to form return light;
the optical property comprises an optical power or intensity of the return light; and
the sensor comprises a spectrometer located separate from the endoscope and configured to measure the optical power or intensity of the return light.

35. The method of claim 34, wherein:
the at least one optical fiber comprises a single optical fiber; and
the method further comprises separating, with a beamsplitter, an optical path of the fiber light from an optical path of the return light.

36. The method of claim 34, wherein:
the at least one optical fiber comprises a first optical fiber and a second optical fiber;
the first optical fiber directs the fiber light distally along the first optical fiber toward the target site; and
the second optical fiber directs the return light proximally along the second optical fiber to the spectrometer.

37. The method of claim 34, wherein the relationship comprises a calibration curve that includes measurements of the optical power or intensity of the light from the target site as a function of distance from the distal end of the at least one optical fiber to the target site, for a plurality of distances from the distal end of the at least one optical fiber to the target site.

38. The method of claim 37, further comprising, with the controller, based at least in part on the calibration curve, dynamically determining a real-time separation between the distal end of the at least one optical fiber and the target site in response to a real-time measured value of the optical power or intensity of the light from the target site.

39. The method of claim 38, further comprising, with the controller, automatically positioning the distal end of the at least one optical fiber, using the calibration curve, to set the real-time separation between the distal end of the at least one optical fiber and the target site to a specified value.

40. The method of claim 38, further comprising, with the controller, providing user feedback representing the real-time separation between the distal end of the at least one optical fiber and the target site.
